(19) United States
(12) Patent Application Publication
(10) Pub. No.: US 2024/0065774 A1
(43) Pub. Date: Feb. 29, 2024

(54) COMPUTER-ASSISTED LOWER-EXTREMITY SURGICAL GUIDANCE

(71) Applicant: Howmedica Osteonics Corp., Mahwah, NJ (US)

(72) Inventors: Jean Chaoui, Lyon (FR); Zachary Michael Korman, Memphis, TN (US)

(21) Appl. No.: 18/271,591

(22) PCT Filed: Jan. 12, 2022
(86) PCT No.: PCT/US2022/012131

§ 371 Date: Jul. 10, 2023

Related U.S. Application Data
(60) Provisional application No. 63/136,502, filed on Jan. 12, 2021.

(51) Int. Cl. A61B 34/20; A61B 17/66; A61B 17/84; A61B 34/10; A61B 90/00

(52) U.S. Cl. CPC A61B 34/20; A61B 17/66; A61B 17/848; A61B 34/10; A61B 90/37; A61B 2017/00725

(57) ABSTRACT

An example method includes obtaining one or more intraoperative images, wherein: a surgical site includes bones that are not substantially exposed through skin of the patient during the surgery, a connected K-wire is attached to one of the bones, an external portion of the connected K-wire is connected to a fixation device that is attached to the patient; performing a registration process system with corresponding positions in a virtual coordinate system; generating a visualization that includes the models of the bones superimposed on the surgical site; based on the changes to the positions of the external portion of the connected K-wire, updating positions of the models of the bones in the visualization to maintain correspondence between positions of the bones and the positions of the models of the bones.

This application claims the benefit of U.S. Provisional Patent Application 63/136,502, filed Jan. 12, 2021, the entire content of which is incorporated by reference.

### TECHNICAL FIELD

This disclosure relates to surgical guidance for orthopedic surgery.

### BACKGROUND

There are many different types of conditions that may require the performance of orthopedic surgery on a patient's foot. For example, a surgeon may perform an orthopedic surgery to correct a bunion or bunionette of a patient's foot. In another example, a surgeon may perform an orthopedic surgery to address a Charcot foot condition of a patient. Orthopedic foot surgery is complicated because there are many bones in the human foot and orthopedic foot surgery frequently requires manipulating various bones and bone fragments.

### SUMMARY

This disclosure describes a variety of techniques for providing intraoperative surgical assistance for minimally invasive surgeries, such as minimally invasive surgeries on lower extremities. As described in this disclosure, a surgical assistance system may use the positions of K-wires attached to bones at a surgical site to determine positions of the bones. Based on the determined positions of the bones, the surgical assistance system may generate a visualization of the bones superimposed on the surgical site or a representation of the surgical site. The visualization may include virtual imagery. In some examples, the virtual imagery may be presented using augmented reality (AR) visualization, mixed reality (MR) visualization, or another type of extended reality (XR) visualization, e.g., via equipment worn by a surgeon and/or other medical staff. The techniques described in this disclosure may be used independently or in various combinations.

In one example, this disclosure describes a computer-implemented method comprising: obtaining, during a surgery performed on a patient, one or more intraoperative images of a surgical site of the patient, wherein: the surgical site includes a plurality of bones that are not substantially exposed through skin of the patient during the surgery, a connected K-wire is attached to a bone of the plurality of bones, an external portion of the connected K-wire is connected to a fixation device that is attached to the patient substantially external to the skin of the patient, and the connected K-wire includes a respective external portion that extends outside the skin of the patient, generating models of the bones and the connected K-wire; determining, based at least in part on the one or more intraoperative images, one or more positions on the external portion of the connected K-wire in a real-world coordinate system; performing a registration process that generates registration data for mapping the positions on the external portion of the connected K-wire in the real-world coordinate system with corresponding positions of a model of the connected K-wire in a virtual coordinate system; generating a visualization that includes the models of the bones superimposed on the surgical site; detecting changes to the positions of the external portion of the connected K-wire, and based on the changes to the positions of the external portion of the connected K-wire, updating positions of the models of the bones in the visualization to maintain correspondence between positions of the bones and the positions of the models of the bones.

In another example, this disclosure describes a computing system comprising: a memory; and processing circuitry configured to: obtain, during a surgery performed on a patient, one or more intraoperative images of a surgical site of the patient, wherein: the surgical site includes a plurality of bones that are not substantially exposed through skin of the patient during the surgery, a connected K-wire is attached to a bone of the plurality of bones, an external portion of the connected K-wire is connected to a fixation device that is attached to the patient substantially external to the skin of the patient, and the connected K-wire includes a respective external portion that extends outside the skin of the patient, generate models of the bones and the connected K-wire; determine, based at least in part on the one or more intraoperative images, one or more positions on the external portion of the connected K-wire in a real-world coordinate system; perform a registration process that generates registration data for mapping the positions on the external portion of the connected K-wire in the real-world coordinate system with corresponding positions of a model of the connected K-wire in a virtual coordinate system; generate a visualization that includes the models of the bones superimposed on the surgical site; detect changes to the positions of the external portion of the connected K-wire; and based on the changes to the positions of the external portion of the connected K-wire, update positions of the models of the bones in the visualization to maintain correspondence between positions of the bones and the positions of the models of the bones.

In other examples, this disclosure describes a computing system comprising means for performing the methods of this disclosure. Moreover, this disclosure describes a computer-readable storage medium having instructions stored thereon that, when executed, cause a computing system to perform the methods of this disclosure.

The details of various examples of the disclosure are set forth in the accompanying drawings and the description below. Various features, objects, and advantages will be apparent from the description, drawings, and claims.

### BRIEF DESCRIPTION OF THE DRAWINGS

FIG. 1 is a block diagram of a surgical assistance system according to an example of this disclosure.

FIG. 2 is a schematic representation of a Mixed Reality (MR) visualization device for use in the surgical assistance system of FIG. 1, according to an example of this disclosure.

FIG. 3 is a block diagram illustrating example components of an intraoperative guidance system (IGS), in accordance with one or more techniques of this disclosure.

FIG. 4 is a conceptual diagram illustrating an example foot of a patient with a fixation device and K-wires, in accordance with one or more techniques of this disclosure.

FIG. 5 is a conceptual diagram illustrating an example visualization of the foot of FIG. 4 with superimposed bone models, in accordance with one or more techniques of this disclosure.

FIG. 6 is a conceptual diagram of a medical image of a foot with a fixation device comprising calibration objects, in accordance with one or more techniques of this disclosure.

FIG. 7 is a flowchart illustrating an example operation of the IGS, in accordance with one or more techniques of this disclosure.

### DETAILED DESCRIPTION

Various types of orthopedic surgeries may be performed to correct foot conditions and other orthopedic conditions. For example, surgeon may perform orthopedic surgeries to correct bunions, bunionettes, Charcot foot conditions, traumatic foot and ankle injuries, and other conditions. At least because a patient's mobility may be restricted following foot surgery, it is desirable to minimize recovery time. Accordingly, it may be desirable to use minimally invasive surgical (MIS) techniques when performing foot surgeries. For instance, when using MIS techniques, a surgeon may only need to create incisions that are a few millimeters (mm) in length. In contrast, traditional bunion surgeries and Charcot foot surgeries frequently involve creating incisions that are several centimeters in length.

However, during surgery, it may be difficult for a surgeon to know exactly where various bones and surgical tools are located and oriented within the foot when MIS techniques are used. For example, it may be difficult for a surgeon to determine exactly how one bone is oriented relative to another bone because the surgeon may be unable to see both of the bones through the very small incision in the patient's foot. To compensate, surgeons frequently take multiple fluoroscopic images of the patient's foot during the foot surgery. The surgeon can use these fluoroscopic images to determine the positions of bones and surgical instruments. However, taking each additional fluoroscopic image causes the patient and/or surgeon to be exposed to additional x-ray radiation, which may have long-term health consequences. Additionally, it takes valuable time for the surgeon to take the fluoroscopic images during the foot surgery.

This disclosure describes techniques that may address one or more of these issues. As described in this disclosure, a surgical assistance system may obtain, during a surgery performed on a patient, one or more intraoperative images of a surgical site of the patient. The surgical site includes a plurality of bones that are not substantially exposed through skin of the patient during the surgery. At least one K-wire of a plurality of K-wires is attached to each bone of the plurality of bones. A K-wire, sometimes referred to as a Kirschner wire, typically is used as stabilization wire or pin in orthopedic surgery. Each of the K-wires includes a respective external portion that extends outside the skin of the patient. The K-wires include at least one connected K-wire. The connected K-wire is a K-wire that has an external portion that is connected to a fixation device that is attached to the patient substantially external to the skin of the patient. Thus, one end of the connected K-wire may be attached to a bone of the patient and another end of the connected K-wire extends outside the patient. K-wires may have various thicknesses and rigidities.

Furthermore, the surgical assistance system may determine, based at least in part on the one or more intraoperative images, 3-dimensional (3D) positions of the K-wires in a virtual coordinate system. The surgical assistance system may also determine positions on the external portions of the plurality of K-wires in a real-world coordinate system. Additionally, the surgical assistance system may perform the registration process that generates registration data for mapping the positions on the external portions of the plurality of K-wires in the real-world coordinate system with corresponding positions among the 3D positions of the K-wires in the virtual coordinate system.

The surgical assistance system may present a visualization that includes the models of the bones superimposed on the surgical site. This visualization may be an augmented reality (AR) visualization, a mixed reality (MR) visualization, or another type of extended reality visualization. The surgical assistance system may detect changes to the positions of the external portions of the K-wires. Based on the changes to the positions of the external portions of the K-wires, the surgical assistance system may automatically move positions of the models of the bones in the visualization to maintain correspondence between positions of the bones and the positions of the models of the bones.

Because the surgeon is able to view the visualization and the visualization is updated as the bones move within the patient, it may be possible for the surgeon to perform a minimally invasive surgery on the patient without taking as many fluoroscopic images during the surgery. This may lead to less radiation exposure and may lead to faster surgery times. In other words, with a surgical assistance system as described in this disclosure, the surgery may be completed with enhanced guidance and reduce exposure to radiation.

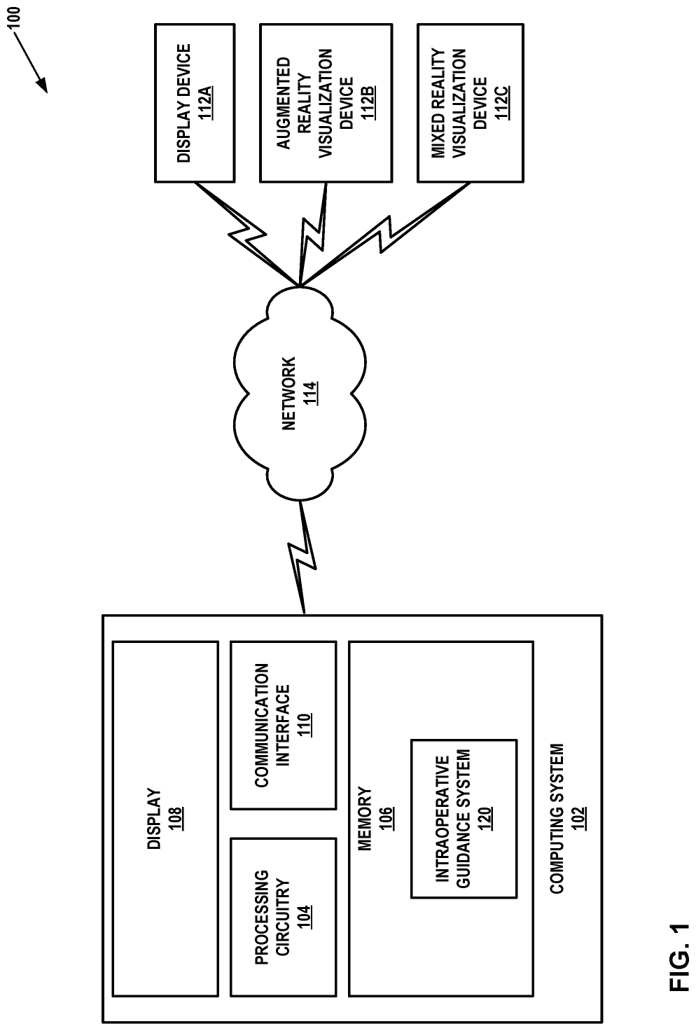

FIG. 1 is a block diagram illustrating an example surgical assistance system 100 that may be used to implement the techniques of this disclosure. In the example of FIG. 1, surgical assistance system 100 includes a computing system 102. Computing system 102 is an example of a computing system configured to perform one or more example techniques described in this disclosure. Computing system 102 may include various types of computing devices, such as server computers, personal computers, smartphones, laptop computers, tablet computers, and other types of computing devices. Computing system 102 includes processing circuitry 104, memory 106, a display 108, and a communication interface 110. Display 108 may be optional, such as in examples where computing system 102 comprises a server computer.

Examples of processing circuitry 104 include one or more microprocessors, digital signal processors (DSPs), application specific integrated circuits (ASICs), field programmable gate arrays (FPGAs), hardware, or any combinations thereof. In general, processing circuitry 104 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable.

Processing circuitry 104 may include arithmetic logic units (ALUs), elementary function units (EFUs), digital circuits, analog circuits, and/or programmable cores, formed from programmable circuits. In examples where the operations of processing circuitry 104 are performed using software executed by the programmable circuits, memory 106 may store the object code of the software that processing circuitry 104 receives and executes, or another memory within processing circuitry 104 (not shown) may store such instructions. Examples of the software include software designed for surgical planning and/or surgical assistance. Processing circuitry 104 may perform the actions ascribed in this disclosure to computing system 102.

Memory 106 may store various types of data used by processing circuitry 104. For example, memory 106 may store data describing 3D models of various anatomical structures, including morbid (pathological) and predicted premorbid (non-pathological) anatomical structures, such as a bone.

Memory 106 may be formed by any of a variety of memory devices, such as dynamic random access memory (DRAM), including synchronous DRAM (SDRAM), magnetoresistive RAM (MRAM), resistive RAM (RRAM), hard disk drives, optical discs, or other types of non-transitory computer-readable media. Examples of display 108 may include a liquid crystal display (LCD), a plasma display, an organic light emitting diode (OLED) display, or another type of display device.

Communication interface 110 allows computing system 102 to output data and instructions to and receive data and instructions from one or more of a display device 112A, an augmented reality (AR) visualization device 112B, a mixed reality (MR) visualization device 112C and/or other devices via a network 114. This disclosure may refer to display device 112A, AR visualization device 112B, and MR visualization device 112C collectively as output devices 112. Communication interface 110 may comprise hardware circuitry that enables computing system 102 to communicate (e.g., wirelessly or using wires) to other computing systems and devices, such as one or more of output devices 112. In some examples, only one of output devices 112 is used during a surgery or is present at all in surgical assistance system 100. Network 114 may include various types of communication networks including one or more wide-area networks, such as the Internet, local area networks, and so on. In some examples, network 114 may include wired and/or wireless communication links.

Display device 112A may be a device that includes a display screen. For example, display device 112A may be or include a computer monitor, television set, or other type of device. Display device 112A may be positioned within an operating room. For instance, display device 112A may be mounted to a wall or ceiling of the operating room, e.g., via a fixed or adjustable arm. In some examples, an assistant may hold display device 112A for viewing by a surgeon.

AR visualization device 112B may be a device that includes a display screen. For example, AR visualization device 112B may be a tablet computer, a laptop computer, a smartphone, a personal computer having a connected display screen, or other type of device. In some examples, AR visualization device 112B is worn on the head of the surgeon. In other examples, AR visualization device 112B is not head-worn. AR visualization device 112B may present AR visualizations to the surgeon during surgery.

MR visualization device 112C may include wearable devices that may be worn by a surgeon during a surgery. MR visualization device 112C may present MR visualization to the surgeon during surgery. In some examples, AR visualization device 112C and/or MR visualization device 112C may be Microsoft HOLOLENS™ headsets, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses.

Furthermore, in the example of FIG. 1, memory 106 may include computer-readable instructions that, when executed by processing circuitry 104, cause computing system 102 to provide an intraoperative guidance system (IGS) 120. In some examples, some or all of the instructions of IGS 120 are stored on AR visualization device 112B, MR visualization device 112C and/or executed by processing circuitry of AR visualization device 112B and/or MR visualization device 112C. For ease of explanation, this disclosure may simply describe actions performed by computing system 102 and/or one or more of output devices 112 when processing circuitry 104 and/or processing circuitry of one or more of output devices 112 executes instructions of IGS 120 as being performed by IGS 120.

One or more users may use IGS 120 during in an intraoperative phase (i.e., during performance of a surgery). For example, IGS 120 may generate visualizations (e.g., for presentation on one or more of output devices 112) that may help a user determine locations of bones during a surgery.

In some examples, IGS 120 may help the one or more users execute a surgical plan that may be customized to an anatomy of interest of a patient. The surgical plan may include a 3-dimensional virtual model that corresponds to the anatomy of interest of the patient and 3-dimensional models of one or more prosthetic components matched to the patient to repair the anatomy of interest or selected to repair the anatomy of interest. The surgical plan may include 3-dimensional virtual models to guide a user in performing the surgical procedure, e.g., in preparing bone surfaces or tissue and placing implantable prosthetic hardware relative to such bone surfaces or tissue. In accordance with one or more techniques of this disclosure, the surgical plan may also include information regarding trajectories for inserting screws, pins, or other items into a bone of the patient.

A surgical plan, such as a surgical plan generated by the BLUEPRINT™ system produced by Wright Medical NV or another surgical planning platform, may include a variety of information regarding a surgical procedure. For example, a surgical plan may include information regarding steps to be performed on a patient by a user, such as a surgeon. Example steps may include, for example, bone or tissue preparation steps and/or steps for selection, modification and/or placement of implant components. Furthermore, information in a surgical plan may include, in various examples, dimensions, shapes, angles, surface contours, and/or orientations of implant components to be selected or modified by users, dimensions, shapes, angles, surface contours and/or orientations to be defined in bone or tissue by the user in bone or tissue preparation steps, and/or positions, axes, planes, angle and/or entry points defining placement of implant components by the user relative to patient bone or tissue. Information such as dimensions, shapes, angles, surface contours, and/or orientations of anatomical features of the patient may be derived from imaging (e.g., x-ray, CT, MRI, ultrasound or other images), direct observation, or other techniques.

IGS 120 may be configured to cause display 108 and/or output devices 112 to display virtual guidance including one or more virtual guides for performing work on a portion of a patient's anatomy. For instance, IGS 120 may cause display 108 to display virtual guidance, such as 3-dimensional virtual models of bones and other virtual objects, on display 108 during a preoperative planning phase of a surgical procedure. IGS 120 may cause MR visualization device 112C to present an MR scene that includes virtual guidance during an intraoperative phase (i.e., during performance of) the surgical procedure.

When IGS 120 causes MR visualization device 112C to present an MR scene, a user of MR visualization device 112C may be able to view real-world objects along with virtual objects. For instance, the user of MR visualization device 112C may be able to see objects in a real-world environment, such as a surgical operating room. In this disclosure, the terms real and real-world may be used in a similar manner. The real-world objects viewed by the user in the real-world scene may include the patient's actual, real anatomy, such as an actual bone, exposed during a surgical procedure.

MR visualization device 112C may be a head-mounted MR visualization device and the user of MR visualization device 112C may view real-world objects via a see-through (e.g., transparent) screen, such as see-through holographic lenses, of MR visualization device 112C and also see virtual guidance that appear to be projected on the screen or within the real-world scene, such that the MR guidance object(s) appear to be part of the real-world scene, e.g., with the virtual objects appearing to the user to be integrated with the actual, real-world scene. For example, the virtual guidance may be projected on the screen of a MR visualization device 112C, such that the virtual guidance is overlaid on, and appears to be placed within, an actual, observed view of the patient's actual bone viewed by the user through the transparent screen, e.g., through see-through holographic lenses. Hence, in this example, the virtual guidance may be a virtual 3D object that appears to be part of the real-world environment, along with actual, real-world objects.

In this disclosure, the term “mixed reality” (MR) refers to the presentation of virtual objects such that a user sees images that include both real, physical objects and virtual objects. Virtual objects may include text, 2-dimensional surfaces, 3-dimensional models, or other user-perceptible elements that are not actually present in the physical, real-world environment in which the virtual objects are presented as coexisting. In addition, virtual objects described in various examples of this disclosure may include graphics, images, animations or videos, e.g., presented as 3D virtual objects or 2D virtual objects. Virtual objects may also be referred to as virtual elements. Such virtual elements may or may not be analogs of real-world objects.

The Microsoft HOLOLENS™ headset, available from Microsoft Corporation of Redmond, Washington, is an example of an MR device that includes see-through holographic lenses that permit a user to view real-world objects through the lens and concurrently view projected 3D holographic objects. The Microsoft HOLOLENS™ headset, and similar waveguide-based visualization devices, are examples of MR visualization devices that may be used in accordance with some examples of this disclosure. Some holographic lenses may present holographic objects with some degree of transparency through see-through holographic lenses so that the user views real-world objects and virtual, holographic objects. In some examples, some holographic lenses may, at times, completely prevent the user from viewing real-world objects and instead may allow the user to view entirely virtual environments. The term mixed reality may also encompass scenarios where one or more users are able to perceive one or more virtual objects generated by holographic projection. In other words, “mixed reality” may encompass the case where a holographic projector generates holograms of elements that appear to a user to be present in the user's actual physical environment.

In some examples, in mixed reality, the positions of some or all presented virtual objects are related to positions of physical objects in the real world. For example, a virtual object may be tethered to a table in the real world, such that the user can see the virtual object when the user looks in the direction of the table but does not see the virtual object when the table is not in the user's field of view. In some examples, in mixed reality, the positions of some or all presented virtual objects are unrelated to positions of physical objects in the real world. For instance, a virtual item may always appear in the top right of the user's field of vision, regardless of where the user is looking.

A surgeon may use IGS 120 during a surgery. As described herein, IGS 120 may obtain, during a surgery performed on a patient, one or more intraoperative images of a surgical site of the patient. Example types of intraoperative images may include fluoroscopic images, x-ray images, ultrasonic images, and other types of images that may be captured during performance of a surgery. The surgical site includes a plurality of bones that are not substantially exposed through the skin of the patient during the surgery. In this disclosure, references to “bones” may apply to complete bones or bone fragments (e.g., parts of a bone fully or partially separated from a complete bone prior to or during surgery). In examples where the surgery is a bunion or bunionette surgery, the plurality of bones may include two or more metatarsals, proximal phalanges, middle phalanges, distal phalanges, cuneiform bones, cuboid bones, or other bones. In examples where the surgery is a Charcot surgery, the plurality of bones may include two or more of any of the bones of the foot, the fibula, or tibia. In other examples, the bones may include the calcaneus or fragments thereof. In other examples, the plurality of bones may include the tibia, fibula, or fragments thereof.

At least one K-wire of a plurality of K-wires is attached to each bone of the plurality of bones. For instance, in a Chevron bunion surgery, a first K-wire may be attached to a proximal fragment of the patient's first metatarsal and a second K-wire may be attached to a distal fragment of the patient's first metatarsal. In the Chevron surgery, the surgeon may perform a lateral displacement of the distal fragment of the first metatarsal bone after cutting the metatarsal bone.

In another example, in an Akin procedure, a first K-wire may be attached to the patient's a proximal fragment of the patient's first proximal phalanx and a second K-wire may be attached to a distal fragment of the patient's first proximal phalanx. In this example, the first proximal phalanx may be partially severed to form the proximal and the distal fragments of the patient's first proximal phalanx. In the Akin procedure, the surgeon may laterally rotate the distal fragment about a cortical bridge between the proximal and distal fragments of the first proximal phalanx, resulting in a medial translation and an inversion rotation of the distal portion until the newly created surfaces of the proximal and distal fragments touch each other. Other types of applicable surgeries may include an Akin surgery, a medial displacement calcaneal osteotomy (MDCO), a minimally invasive metatarsal osteotomy (DMMO), a transverse first metatarsal osteotomy, a transverse fifth metatarsal osteotomy, or other types of surgeries.

In an example where the surgery is a Lapidus surgery, a first K-wire may be attached to the patient's first metatarsal and a second K-wire may be attached to the patient's first cuneiform bone. In an example where the surgery is a Charcot surgery, K-wires may be attached to one or more of the metatarsals, calcaneus, cuneiform bones, and so on. In another example, the surgery may be a total ankle replacement surgery. It is noted that K-wires may or may not be attached to all bones involved in the surgery. In some examples, one or more K-wires may be attached to bones that are not directly involved in the surgery.

Each of the K-wires includes a respective external portion that extends outside the skin of the patient. The K-wires include at least one connected K-wire. An external portion of the connected K-wire is connected to a fixation device that is attached to the patient substantially external to the skin of the patient. The fixation device may be configured to remain at a fixed position relative to the surgical site. For instance, with respect to a surgery that involves the midfoot or forefoot, the fixation device may include a c-shaped clamp structure that is positioned around the patient's foot and secured (e.g., tightened) so as not to shift positions relative to the patient's foot. The external portion of a K-wire is any portion of the K-wire that extends outside the skin of the patient.

IGS 120 may generate models of the bones based at least in part on the one or more intraoperative images. For example, IGS 120 may generate 3D models of the bones. Additionally, IGS 120 may determine, based at least in part on the one or more intraoperative images, positions on the external portions of the plurality of K-wires in a real-world coordinate system. For example, IGS 120 may use a Simultaneous Localization and Mapping (SLAM) algorithm to determine the positions of the external portions of the plurality of K-wires in the real-world coordinate system. The external portions of the K-wires are portions of the K-wires that are outside of the skin of the patient.

IGS 120 may perform a registration process that generates registration data for mapping the positions on the external portions of the plurality of K-wires in the real-world coordinate system with corresponding positions among the 3D positions of the K-wires in a virtual coordinate system. Positions of the 3D models of the bones may be expressed in terms of coordinates in the virtual coordinate system. Because IGS 120 has data indicating the spatial relationship between the K-wires and bones (e.g., based on intraoperative medical images of the K-wires and bones), because the external portions of the K-wires may have fixed spatial relationships relative to the bones, and because IGS 120 has determined the positions of the external portions of the K-wires, the registration data generated by performing the registration process may enable IGS 120 to determine a spatial relationship between the models of the bones and the actual bones of the patient. In some examples, there may be some curvilinearity in one or more of the K-wires. Accordingly, in some examples, IGS 120 may detect and compensate for the curvilinearity of the K-wires when determining the positions of the bones. In some examples, curvilinearity of a K-wire occurs when a bone to which the K-wire is attached does not move proportionally to an amount that an external portion of the K-wire is moved (e.g., by manipulating features of the fixation device). In such examples, where a manipulating feature of the fixation device is near an outer surface of the fixation device, IGS 120 may use data from one or more sensors in the fixation device to detect curvilinearity between the outer surface of the fixation device and the inner surface of the fixation device. In some examples, IGS 120 may generate indications (e.g., alerts, suggestions, etc.) indicating when there is excessive curvilinearity in a K-wire. The indications may, in some instances, indicate actions that may be performed to mitigate risks associated with excessive bending of a K-wire.

Accordingly, IGS 120 may generate a visualization that includes the models of the bones superimposed on the surgical site. For example, IGS 120 may present an AR visualization in which AR visualization device 112B shows the models of the bones superimposed on an image (or a series of live images) of the surgical site of the patient. For instance, IGS 120 may generate an AR visualization for display on a computer screen (which may or may not be head-mounted) that also displays images of the real-world environment. In another example, IGS 120 may present an MR visualization in which MR visualization device 112C shows the models of the bones superimposed on the actual surgical site of the patient. For instance, IGS 120 may generate a MR visualization for display on a head-mounted MR visualization device configured to allow a user to view the MR visualization and directly see a real-world environment. Thus, in these examples, the AR or MR visualization may have an appearance akin to the surgeon having “x-ray vision” in that the surgeon can virtually see through the skin of the patient to the bones of the patient.

Furthermore, IGS 120 may detect changes to the positions of the external portions of the K-wires. For example, a surgeon may use one or more of the K-wires to adjust a position of a bone. For instance, the surgeon may change an external position of the connected K-wire, and thereby change a position of the bone to which the connected K-wire is attached, by adjusting a knob, ratchet, screw, cam, or other arrangement of the fixation device. The fixation device may ensure that the connected K-wire and hence the bone attached to the connected K-wire remains at a fixed position relative to the rest of bones in the surgical site. For example, if the surgeon uses the fixation device to change a position of a first metatarsal relative to the first cuneiform bone by moving a K-wire connected to the first metatarsal, the first metatarsal will remain at that position relative to the first cuneiform bone until the surgeon performs further actions to move one or more of the bones. Based on the changes to the positions of the external portions of the K-wires, IGS 120 may update positions of the models of the bones in the visualization to maintain correspondence between positions of the bones and the positions of the models of the bones. Thus, the positions of models of the bones shown in the visualization may continue to correspond the positions of the real bones of the patient.

In some examples, the surgery may involve attaching a limb reconstruction frame (LRF) to a limb of a patient, such as an arm or leg of the patient. An LRF may be used to stabilize and reposition bones of a limb during and/or after a surgery. For example, if a tibia of a patient is fractured into two or more fragments, e.g., due to trauma, an LRF may be used in a surgery to hold the fragments at the correct positions for healing. The LRF may include one or more frame members connected by truss members. In some examples, the frame members of the LRF are rings, arcs, horseshoe-shaped objects, or have other shapes. The truss members hold the frame members at consistent locations relative to one another. The truss members and/or frame members may include features, such as set screws, cams, etc, that may enable the movement of the frame members relative to one another in a controlled way. External portions of K-wires may be connected to the frame members of an LRF. Internal portions of the K-wires may be attached to bones of the patient. In some examples, the K-wires may serve to hold two or more bones together. In accordance with one or more techniques of this disclosure, IGS 120 may determine, based at least in part on one or more intraoperative images, one or more positions on the external portions of connected K-wires (e.g., K-wires connected to the LRF) in a real-world coordinate system. IGS 120 may also perform a registration process that generates registration data for mapping the positions on the external portions of the connected K-wires in the real-world coordinate system with corresponding positions of models of the connected K-wires in a virtual coordinate system. IGS 120 may generate a visualization that includes the models of the bones superimposed on the surgical site. IGS 120 may detect changes to the positions of the external portions of the connected K-wires. Based on the changes to the positions of the external portions of the connected K-wires, IGS 120 may update positions of the models of the bones in the visualization to maintain correspondence between positions of the bones and the positions of the models of the bones. In this example and elsewhere in this disclosure, a K-wire may be a threaded pin.

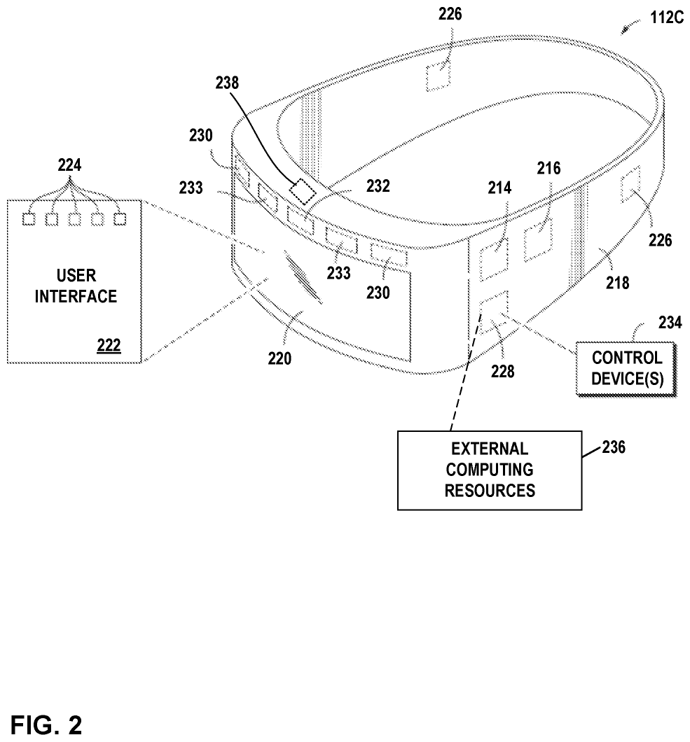

FIG. 2 is a schematic representation of MR visualization device 112C for use in surgical assistance system 100 of FIG. 1, according to an example of this disclosure. As shown in the example of FIG. 2, MR visualization device 112C can include a variety of electronic components found in a computing system, including one or more processor(s) 214 (e.g., microprocessors or other types of processing units) and memory 216 that may be mounted on or within a frame 218. Although the example of FIG. 2 illustrates MR visualization device 112C as a head-wearable device, MR visualization device 112C may have other forms and form factors. For instance, in some examples, MR visualization device 112C may be a handheld smartphone or tablet.

In the example of FIG. 2, MR visualization device 112C includes a transparent screen 220 that is positioned at eye level when MR visualization device 112C is worn by a user. In some examples, screen 220 may include one or more liquid crystal displays (LCDs), organic light emitting diode (OLED) displays, or other types of display screens on which images are perceptible to a user who is wearing or otherwise using MR visualization device 112C. In some examples, MR visualization device 112C can operate to project 3D images onto the user's retinas using techniques known in the art.

In some examples, screen 220 may include see-through holographic lenses, which are sometimes referred to as waveguides. The see-through holographic lenses permit a user to see real-world objects through (e.g., beyond) the see-through holographic lenses and also see holographic imagery projected into the see-through holographic lenses and from there onto the user's retinas. The holographic imagery may be projected by displays, such as liquid crystal on silicon (LCoS) display devices, which are sometimes referred to as light engines or projectors, operating as a holographic projection system 238 within MR visualization device 112C. Hence, in some examples, MR visualization device 112C can project 3D images onto the user's retinas via screen 220. In this manner, MR visualization device 112C may be configured to present a virtual image to a user within a real-world view observed through screen 220, e.g., such that the virtual image appears to form part of the real-world environment. In some examples, MR visualization device 112C may be a Microsoft HOLOLENS™ headset, available from Microsoft Corporation, of Redmond, Washington, USA, or a similar device, such as, for example, a similar MR visualization device that includes waveguides.

Furthermore, in the example of FIG. 2, MR visualization device 112C may generate a user interface (UI) 222 that is visible to the user, e.g., as holographic imagery projected into see-through holographic lenses as described above. UI 222 may include a variety of selectable widgets 224 that allow the user to interact with IGS 120.

MR visualization device 112C also may include other components. For example, MR visualization device 112C may include one or more speakers or other sensory devices 226 that may be positioned adjacent the user's ears. Sensory devices 226 may convey audible information or other perceptible information (e.g., vibrations) to assist the user of MR visualization device 112C. MR visualization device 112C can also include a transceiver 228 to connect MR visualization device 112C to network 114, such as via a wired or wireless communication channel.

MR visualization device 112C may also include a variety of sensors to collect sensor data, such as one or more optical camera(s) 230 (or other optical sensors) and one or more depth camera(s) 232 (or other depth sensors), mounted to, on or within frame 218. In some examples, the optical sensor(s) 230 are operable to scan the geometry of the physical environment in which user of computing system 102 is located (e.g., an operating room) and collect two-dimensional (2D) optical image data (either monochrome or color). Depth sensor(s) 232 are operable to provide 3D image data, such as by employing time of flight, stereo or other known or future-developed techniques for determining depth and thereby generating image data in three dimensions. Other sensors of MR visualization device 112C may include motion sensors 233 (e.g., Inertial Mass Unit (IMU) sensors, accelerometers, etc.) to assist with tracking movement.

IGS 120 (FIG. 1) may process sensor data so that IGS 120 may define geometric, environmental, textural, etc. landmarks (e.g., corners, edges or other lines, walls, floors, objects) in the user's environment and detect movements within the user's environment. As an example, IGS 120 may combine or fuse various types of sensor data so that the user of MR visualization device 112C is able to perceive virtual objects that can be positioned or fixed and/or moved within an MR scene. When a virtual object is fixed in the MR scene, the user can walk around the virtual object, view the virtual object from different perspectives, and manipulate the virtual object within the scene using hand gestures, voice commands, gaze line (or direction) and/or other control inputs. As another example, IGS 120 may process the sensor data so that the user can position a virtual object (e.g., a 3-dimensional bone model) on an observed physical object in the user's environment (e.g., a surface, the patient's real bone, etc.) and/or orient the virtual object with other virtual objects presented in the MR scene. In some examples, IGS 120 may process the sensor data so that the user can position and fix virtual objects representing aspects of a surgical plan onto one or more surfaces, such as one or more walls of an operating room. Furthermore, in some examples, IGS 120 may use the sensor data to recognize surgical instruments and the positions and/or locations of those surgical instruments.

MR visualization device 112C may include one or more processors 214 and memory 216, e.g., within frame 218 of MR visualization device 112C. In some examples, one or more external computing resources 236 process and store information, such as sensor data, instead of or in addition to processor(s) 214 of MR visualization device 112C and memory 216 of MR visualization device 112C. Computing system 102 may include external computing resources 236. For instance, external computing resources 236 may include processing circuitry 104 (FIG. 1) and/or memory 106 (FIG. 1) of computing system 102. In this way, processor(s) 214 and memory 216 of MR visualization device 112C may perform data processing and storage and/or some of the processing and storage requirements may be offloaded from MR visualization device 112C. Hence, in some examples, operation of MR visualization device 112C may, in some examples, be controlled in part by a combination of one or more processors 214 within MR visualization device 112C and processing circuitry 104 external to MR visualization device 112C. In some examples, processor(s) 214 and memory 216 of MR visualization device 112C may provide sufficient computing resources to process the sensor data collected by cameras 230, 232 and motion sensors 233.

In some examples, IGS 120 may process the sensor data using a Simultaneous Localization and Mapping (SLAM) algorithm, or other algorithm for processing and mapping 2D and 3D image data and tracking the position of MR visualization device 112C in the 3D scene. In some examples, image tracking may be performed using sensor processing and tracking functionality provided by the Microsoft HOLOLENS™ system, e.g., by one or more sensors and processors 214 within a MR visualization device 112C substantially conforming to the Microsoft HOLOLENS™ device or a similar MR visualization device.

In some examples, surgical assistance system 100 can also include user-operated control device(s) 234 that allow the user to operate surgical assistance system 100 (e.g., computing system 102 of surgical assistance system 100) and/or MR visualization device 112C. As examples, control device(s) 234 may include a microphone, a touch pad, a control panel, a motion sensor or other types of control input devices with which the user can interact.

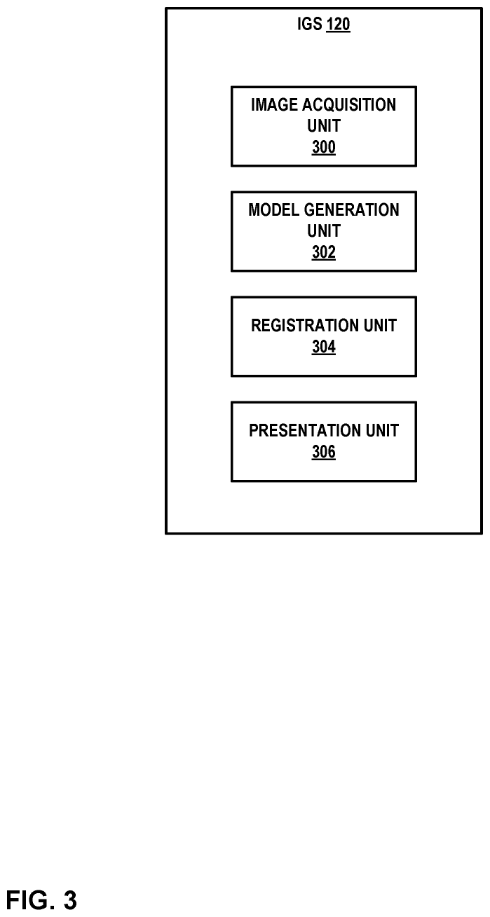

FIG. 3 is a block diagram illustrating example components of IGS 120, in accordance with one or more techniques of this disclosure. In the example of FIG. 3, IGS 120 includes an image acquisition unit 300, a model generation unit 302, a registration unit 304, and a presentation unit 306. In other examples, IGS 120 may include more, fewer, or different units. One or more of the units in FIG. 3 may be logical, and do not necessarily correspond one-to-one to implemented code. In some examples, one or more of the units may be distinct circuit blocks (fixed-function or programmable), and in some examples, one or more of the units may be integrated circuits.

Image acquisition unit 300 is configured to obtain images of a surgical site of a patient, such as a foot or ankle of the patient. The images may include preoperative images (i.e., images generated before beginning the surgery) and intraoperative images (i.e., images generated after the surgery). The images may include fluoroscopic images, x-ray images, CT images, and/or other types of medical images. In some examples, image acquisition unit 300 obtains one or more of the images from a memory (e.g., memory 106) that stores the images. In some examples, image acquisition unit 300 obtains one or more of the images directly from an imaging device, such as a fluoroscopic imaging machine.

In accordance with one or more techniques of this disclosure, the images of the surgical site may show a plurality of bones at the surgical site. The bones may include complete natural bones or bone fragments. In some examples, these bones are not substantially exposed through the skin of the patient during the surgery. For instance, the bones are not substantially exposed through the skin if the surgeon is not able to determine a full spatial position and orientation of the bones through the skin of the patient by sight of the bone itself. In some instances, the bones may not be exposed at all through the skin of the patient.

The plurality of bones may be or include all of the bones at the surgical site, a subset of bones at the surgical site, or one or more bones surrounding the surgical site. For instance, in an example where the surgical site is the forefoot of the patient, the surgical site may include five metatarsals and fifteen phalanges. However, the plurality of bones may be limited to a subset of the bones at the surgical site (e.g., the first metatarsal and first proximal phalanx) or bones outside the surgical site, such as cuneiform bones or the cuboid bone.

At least one K-wire of a plurality of K-wires may be attached to each bone of the plurality of bones. Accordingly, one or more of the intraoperative images may show the K-wires attached to the bones. Moreover, different intraoperative images may show the K-wires and bones from different angles. This may allow IGS 120 to determine the 3D positions and orientations of the K-wires relative to the bones to which the K-wires are attached. Each of the K-wires has an external portion that extends beyond the skin of the patient.

Model generation unit 302 may generate models of the bones (e.g., 2D or 3D models of the bones). In some examples, model generation unit 302 may generate models of one or more of the bones based on preoperative images. In some examples, model generation unit 302 generates models of one or more of the bones based on intraoperative images. In either case, model generation unit 302 may use intraoperative images to generate models of the K-wires attached to the bones. In some examples, the models of the K-wires may be full 3D virtual objects representing the K-wires. In some examples, the models of the K-wires may be limited to specific points or areas of the K-wires, such as coordinates of an external tip of a K-wire and coordinates of an entry point of the K-wire into a bone of the patient. In some examples, model generation unit 302 may also use intraoperative images to generate a model of the fixation device and/or other surgical items.

Model generation unit 302 may generate a model of a bone in one of a variety of ways. For instance, in some examples, model generation unit 302 may project variations of a shape of a 3D model onto a 2D plane according to a known x-ray setup, e.g., given a relative position of an x-ray (point) source to an x-ray detector. Model generation unit 302 may determine that the shape that maximizes the similarity between the 3D model's projection and the x-ray image(s) to be the best approximation of the true 3D anatomy. Model generation unit 302 may compare the similarity if the projected contours of the 3D model and silhouettes from the reference X-ray. Although contours or silhouettes of 3D anatomical models can be computed very efficiently, the contours or silhouettes may first have to be extracted from the individual patient's x-ray images in a manual or automatic manner. In some examples, model generation unit 302 may project virtual x-rays instead of comparing silhouettes or contours. In such examples, model generation unit 302 may project virtual x-ray images from the 3D models that simulate an x-ray screening according to a clinical setup. Model generation unit 302 may compare the virtual X-ray images directly to the reference X-rays by means of their intensity distribution, taking the bone-interior structures into account. In some examples, model generation unit 302 may use a generative adversarial network (GAN) convolutional neural network (CNN) to generate such 3D model from 2D x-rays.

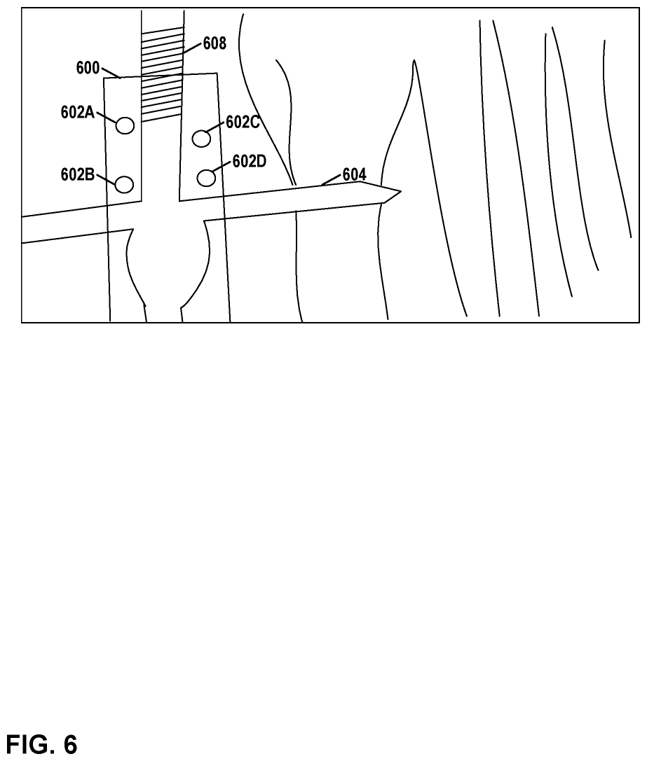

In some examples, registration unit 304 performs a calibration process to determine a size of the bones and/or K-wires. In accordance with one or more techniques of this disclosure, intraoperative images may contain images of the fixation device, which during the surgery, is attached to the surgical site. The fixation device may include calibration objects of the fixation device. The calibration objects are objects that have predetermined sizes and shapes. For instance, one or more of the calibration objects may be a sphere. In some examples, one or more of the calibration objects of the fixation device are embedded within the fixation device. In some examples, one or more of the calibration objects are attached to an outer surface of the fixation device. FIG. 6, which is described in greater detail below, shows an intraoperative image that includes an image of the fixation device with embedded calibration objects.

Registration unit 304 may determine, based at least in part on the one or more intraoperative images, positions on the external portions of the plurality of K-wires in a real-world coordinate system. As part of performing the registration process, surgical assistance system 100 may generate a first point cloud and a second point cloud. The first point cloud includes points corresponding to landmarks on one or more virtual objects, such as a pre-revision model of a bone. The second point cloud includes points corresponding to landmarks on real-world objects. The positions of the external portions of the plurality of K-wires may be included in the second point cloud. Landmarks may be locations on virtual or real-world objects. The points in the first point cloud may be expressed in terms of coordinates in a first coordinate system (i.e., a virtual coordinate system) and the points in the second point cloud may be expressed in terms of coordinates in a second coordinate system (i.e., a real-world coordinate system). Because the virtual objects may be designed with positions that are relative to one another but not relative to any real-world objects, the first and second coordinate systems may be different.

Registration unit 304 may generate the second point cloud using a Simultaneous Localization and Mapping (SLAM) algorithm. By performing the SLAM algorithm, registration unit 304 may generate the second point cloud based on observation data generated by sensors (e.g., depth sensor(s) 232 of MR visualization device 112C) while also tracking a location of MR visualization device 112C. Registration unit 304 may perform one of various implementations of SLAM algorithms, such as a SLAM algorithm having a particular filter implementation, an extended Kalman filter implementation, a covariance intersection implementation, a GraphSLAM implementation, an ORB SLAM implementation, or another implementation. In some examples, registration unit 304 may apply an outlier removal process to remove outlying points in the first and/or second point clouds. In some examples, the outlying points may be points lying beyond a certain standard deviation threshold from other points in the point clouds. Applying outlier removal may improve the accuracy of the registration process.

Furthermore, as part of performing the registration process, the visualization presented by output devices 112 may include a starting-point virtual object. The starting-point virtual object may be a virtual object that corresponds to a real-world object visible to the user, such as an external portion a K-wire. Additionally, registration unit 304 may receive an indication of user input to position the starting-point virtual object such that the starting-point virtual object is generally at a position of the corresponding real-world object. For instance, registration unit 304 may receive an indication of user input to position the 3D model of a K-wire onto the real-world K-wire. Positioning the starting-point virtual object onto the corresponding real-world object may enable registration unit 304 to determine a preliminary spatial relationship between points in the first point cloud and points in the second point cloud. The preliminary spatial relationship may be expressed in terms of translational and rotational parameters.

Next, registration unit 304 may refine the preliminary spatial relationship between points in the first point cloud and points in the second point cloud. For example, registration unit 304 may perform an iterative closest point (ICP) algorithm to refine the preliminary spatial relationship between the points in the first point cloud and the points in the second point cloud. The iterative closest point algorithm finds a combination of translational and rotational parameters that minimize the sum of distances between corresponding points in the first and second point clouds. For example, consider a basic example where landmarks corresponding to points in the first point cloud are at coordinates A, B, and C and the same landmarks corresponding to points in the second point cloud are at coordinates A′, B′, and C′. In this example, the iterative closest point algorithm determines a combination of translational and rotational parameters that minimizes ΔA+ΔB+ΔC, where ΔA is the distance between A and A′, ΔB is the distance between B and B′, and ΔC is the distance between C and C′. To minimize the sum of distances between corresponding landmarks in the first and second point clouds, registration unit 304 may perform following steps:
1. For each point of the first point cloud, determine a corresponding point in the second point cloud. The corresponding point may be a closest point in the second point cloud. In this example, the first point cloud includes points corresponding to landmarks on one or more virtual objects and the second point cloud may include points corresponding to landmarks on real-world objects.
2. Estimate a combination of rotation and translation parameters using a root mean square point-to-point distance metric minimization technique that best aligns each point of the first point cloud to its corresponding point in the second point cloud.
3. Transform the points of the first point cloud using the estimated combination of rotation and translation parameters.
4. Iterate steps 1-3 using the transformed points of the first point cloud. The iteration may continue for a specific number of rounds, until changes between iterations decrease to a specific threshold, or another stopping criteria is reached.
   In this example, after performing an appropriate number of iterations, registration unit 304 may determine rotation and translation parameters that describe a spatial relationship between the original positions of the points in the first point cloud and the final positions of the points in the first point cloud. The determined rotation and translation parameters can therefore express a mapping between the first point cloud and the second point cloud.

Presentation unit 306 may generate and present a visualization that includes the models of the bones superimposed on the surgical site. Presentation unit 306 may generate a non-XR visualization (e.g., a 2-dimensional visualization), an AR visualization, an MR visualization, or another type of visualization.

In some examples, the visualization generated by presentation unit 306 is an AR visualization. AR visualization device 112B may present the AR visualization. In such examples, presentation unit 306 may obtain live image data of the surgical site, e.g., via optical cameras 230 (FIG. 2) or other cameras. The live video images of the surgical site may include images of the external portions of the K-wires. Additionally, presentation unit 306 may obtain depth data to determine positions of the external portions of the K-wires in the real-world coordinate system. For example, presentation unit 306 may use depth data from depth sensor(s) 232 (FIG. 2) to determine the positions of the external portions of the K-wires in the real-world coordinate system. In other examples, presentation unit 306 may use data from multiple optical cameras 230 (FIG. 2) or other cameras to determine the positions of the external portions of the K-wires in the real-world coordinate system. Presentation unit 306 may use the registration data generated by registration unit 304 to convert the positions of the external portions of the virtual models (e.g., of K-wires and bones) from the virtual coordinate system to the real-world coordinate system such that the corresponding points in the K-wires and models of the K-wires are aligned. Presentation unit 306 may then generate a visualization that includes image data showing the models of the bones superimposed onto the video images at positions corresponding to the coordinates in the real-world coordinate system.

In some examples, the visualization generated by presentation unit 306 is an MR visualization. MR visualization device 112C may present the MR visualization. In such examples, presentation unit 306 may obtain depth data (e.g., via depth sensor(s) 232 or optical sensors 230) to determine positions of the external portions of the K-wires in the real-world coordinate system. Presentation unit 306 may then use the registration data generated by registration unit 304 to convert the coordinates of the models of the bones and K-wires from the virtual coordinate system to the real-world coordinate system such that the corresponding points in the K-wires and models of the K-wires are aligned. Presentation unit 306 may then generate a visualization that includes image data of the models of the bones at locations in the MR display that cause the user of MR visualization device 112C to perceive the models of the bones to be superimposed on the real surgical site of the patient at the actual locations of the bones.

In some examples where the visualization is a non-extended reality (XR) visualization, such as a 2-dimensional visualization, display device 112A may display the non-XR visualization. This disclosure uses the term XR to refer to a spectrum of experiences from AR and MR to virtual reality (VR). The process for generating the 2-dimensional visualization may be similar to generating the AR visualization, except that the models of the bones are not displayed in the context of real-world images. For instance, in some examples where the surgical site is the patient's foot, the non-XR visualization may show the bone models relative to an outline of a generic or patient-specific foot instead of live images of the patient's actual foot.

Presentation unit 306 may continue to obtain depth data regarding the positions of external portions of the K-wires during the surgery. Presentation unit 306 may compare the coordinates in the external portions of the K-wires to previous coordinates of the external portions of the K-wires. In this way, presentation unit 306 may detect changes in the positions of the external portions of the K-wires. If there is a difference in the position of a K-wire, presentation unit 306 may update positions of the models of the bones, associated with the K-wires, in the visualization to maintain correspondence between positions of the bones and the positions of the models of the bones.

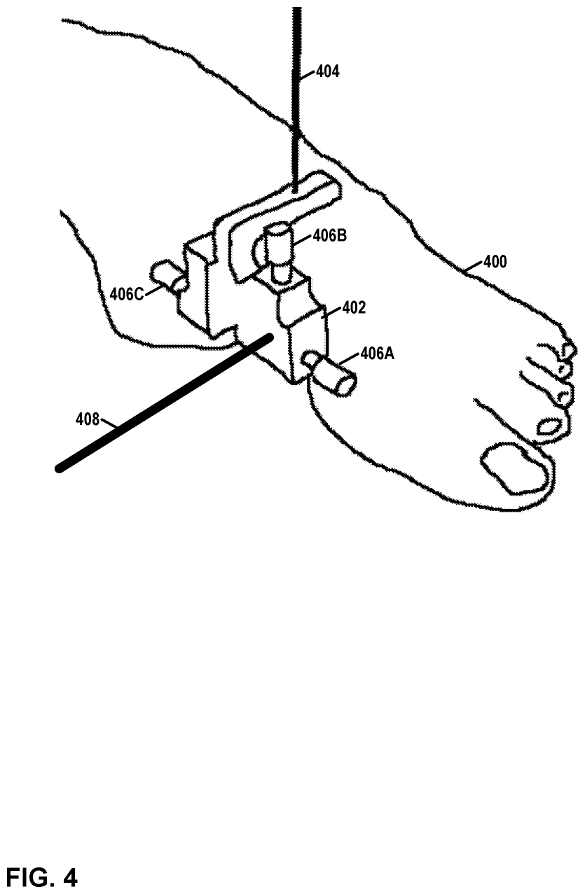

FIG. 4 is a conceptual diagram illustrating an example foot 400 of a patient with a fixation device 402 and K-wires 404, 408, in accordance with one or more techniques of this disclosure. In the example of FIG. 4, fixation device 402 forms a clamp that is fixed during a surgery to foot 400 of a patient. As shown in the example of FIG. 4, K-wire 408 is a connected K-wire that is connected to fixation device 402 and a bone of the patient. Fixation device 402 includes adjustment features 406A, 406B, and 406C (collectively, “adjustment features 406”) that may be used for making adjustments to the position of one or more K-wires (which are not shown in the example of FIG. 4). In the example of FIG. 4, adjustment features 406 are in the form of knobs connected to set screws. In the example of FIG. 4, K-wire 404 may be inserted into a position between bones during a Lapidus procedure.

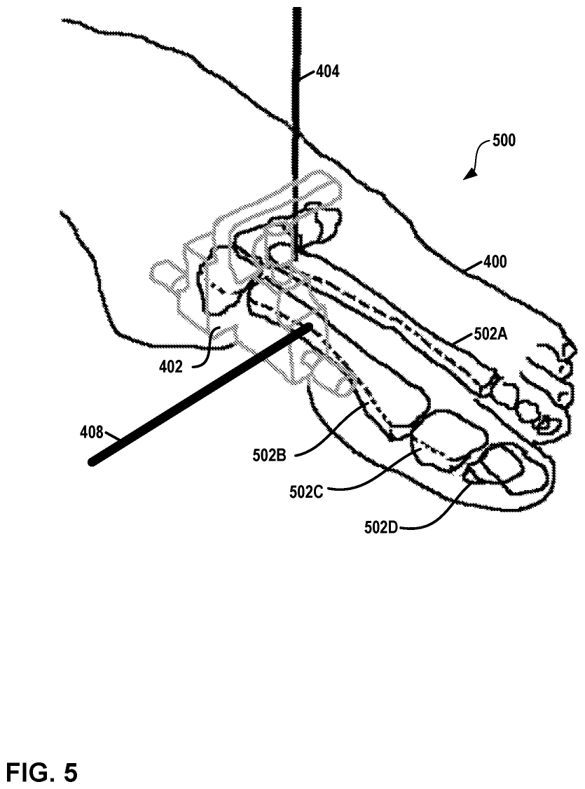

FIG. 5 is a conceptual diagram illustrating an example visualization 500 of the foot 400 of FIG. 4 with superimposed bone models 502A through 502D (collectively, “bone models 502”), in accordance with one or more techniques of this disclosure. For visual clarity, not all the bone models (e.g., models for the second phalanges and cuneiform bone) have reference numbers in FIG. 5. In examples where visualization 500 is a non-XR visualization, foot 400 shown in FIG. 5 may be a generic outline of a foot or a patient-specific outline of the foot. In examples where visualization 500 is an AR visualization, foot 400 shown in FIG. 5 may be a live image of the patient's foot. In examples where visualization 500 is an MR visualization, foot 400 shown in FIG. 5 may actually be the patient's foot, e.g., observed via a see-through lens or lenses.

FIG. 6 is a conceptual diagram of a medical image of a foot with a fixation device 600 comprising calibration objects 602A through 602D, in accordance with one or more techniques of this disclosure. This disclosure may refer to calibration objects 602A through 602D as “calibration objects 602.” In the example of FIG. 6, the medical image is from a superior position and shows a K-wire 604 attached to a first metatarsal 606. Although not shown in the example of FIG. 6, set screw 608 may be attached to a knob. Turning the knob may adjust the position of K-wire 604.

In the example of FIG. 6, calibration objects 602 are physical spheres that have known sizes and positions within fixation device 600. For instance, calibration objects 602 may be metal balls. Calibration objects 602 may appear to be larger or smaller in medical images depending on the distance of calibration objects from a detector that generated the medical images. Because calibration objects 602 have known sizes, image acquisition unit 300 may determine a true scale of the medical image based on a comparison of the known sizes of calibration objects 602 and apparent sizes of calibration objects in the medical image.

Thus, fixation device 600 may include one or more calibration objects 602 of known sizes. The intraoperative images include phantoms (i.e., shadows) caused by calibration objects 602. In some examples, IGS 120 (e.g., image acquisition unit 300 (FIG. 3) or model generation unit 302 of IGS 120) may determine calibration parameters based on a comparison of the known sizes of the one or more calibration objects and apparent sizes of the phantoms caused by the calibration objects in the intraoperative images. The calibration parameters may indicate a relation between the real size of an object in a picture and the apparent size of the object in the picture.

In some examples, IGS 120 (e.g., image acquisition unit 300 (FIG. 3) or model generation unit 302 of IGS 120) may determine calibration parameters based on a comparison of relative positions of the one or more calibration objects within fixation device 600 and apparent positions of the phantoms caused by calibration objects 602 in the intraoperative images. IGS 120 may modify the one or more intraoperative images based on the calibration parameters. For example, IGS 120 may scale the intraoperative images based on the calibration parameters such that the area shown in the intraoperative images has a standard size. In this example, IGS 120 (e.g., model generation unit 302 of FIG. 3) may generate the models of the bones and the models of the K-wires based on the modified one or more intraoperative images.

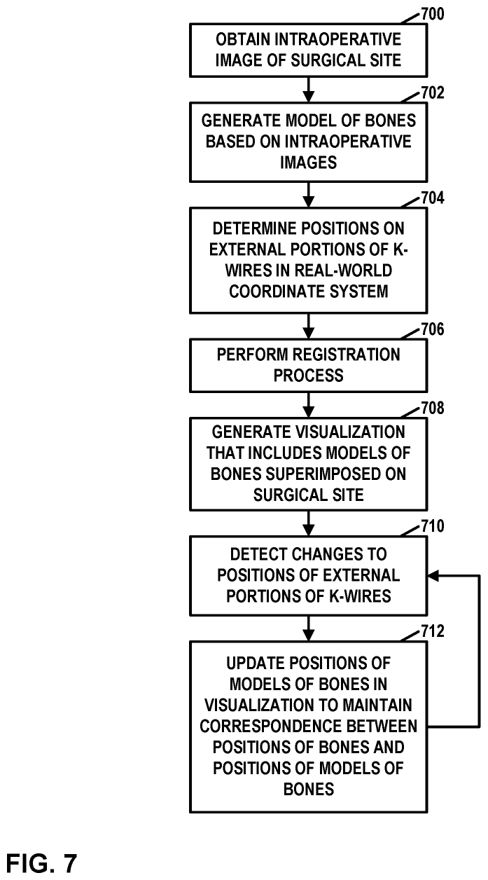

FIG. 7 is a flowchart illustrating an example operation of IGS 120, in accordance with one or more techniques of this disclosure. In the example of FIG. 7, IGS 120 may obtain, during a surgery performed on a patient, one or more intraoperative images of a surgical site of the patient (700). The surgical site includes a plurality of bones that are not exposed or at least not substantially exposed through skin of the patient during the surgery. Furthermore, in some examples, at least one K-wire of a plurality of K-wires is attached to each bone of the plurality of bones, where the plurality of bones may be a subset of bones in the foot, and may represent bones of interest for purposes of the surgical procedure. Each of the K-wires includes a respective external portion that extends outside the skin of the patient. The K-wires include a connected K-wire. An external portion of the connected K-wire is connected to a fixation device that is attached to the patient substantially external to the skin of the patient. The fixation device may include one or more features for moving the connected K-wire, e.g., moving the connected K-wire relative to other ones of the K-wires, and thereby moving pertinent bones.

Furthermore, in the example of FIG. 7, IGS 120 (e.g., model generation unit 302 of IGS 120) may generate models of the bones and one or more of the K-wires, such as the connected K-wire (702). IGS 120 may generate the models in any of a variety of ways. For instance, in one example, to generate the models of the bones, IGS 120 may generate preoperative 3D models of the bones based on preoperative medical images of the surgical site. Additionally, in this example, IGS 120 may generate intraoperative 3D models of the bones based on intraoperative images of the surgical site and 3D models of K-wires based on the intraoperative images of the surgical site. Furthermore, in this example, IGS 120 may align preoperative 3D models of K-wires (including the connected K-wire) with preoperative 3D models of the bones based on comparison of landmarks on the preoperative 3D models of bones and corresponding landmarks on intraoperative 3D models of the bones.

In examples where the preoperative images have higher quality (e.g., in examples where the preoperative images are CT scans), the resulting 3D bone models based on the preoperative images may have been higher quality (e.g., have greater precision, more detail, etc.) than 3D bone models based on the intraoperative images. However, the preoperative images do not show the locations of the K-wires relative to the bones. By aligning the bone models based on the preoperative images with the models based on the intraoperative images, IGS 120 may determine the positions of the K-wires relative to the bone models based on the preoperative images. Thus, IGS 120 may use the bone models based on the preoperative images instead of the bone models based on the intraoperative images for at least some purposes when generating visualizations, while still using the models of the K-wires. In this way, the visualizations may show the bone models with the increased accuracy of the preoperative images. Generating the bone models to the same level of accuracy based on intraoperative images may require generating more intraoperative images, with an associated increased radiation dose. In other examples, IGS 120 may generate the bone models only based on intraoperative images.

IGS 120 (e.g., registration unit 304 of IGS 120) may determine, based at least in part on the one or more intraoperative images, positions on the external portions of one or more of the K-wires (including the external portion of the connected K-wire) in a real-world coordinate system (704). In some examples, IGS 120 may determine, based on video data generated by cameras and/or other sensors of the head-mounted MR visualization device, the positions of the external portions of the K-wires in the real-world coordinate system.

Additionally, IGS 120 (e.g., registration unit 304) may perform a registration process that generates registration data for mapping the positions on the external portions of the plurality of K-wires in the real-world coordinate system with corresponding positions among 3D positions of the K-wires in a virtual coordinate system (706). For instance, IGS 120 may perform a registration process that generates registration data for mapping the positions on the external portions of the connected K-wire in the real-world coordinate system with corresponding positions of 3D positions of the connected K-wire in a virtual coordinate system. An example of the registration process is provided above. In some examples, fiducial markers may be attached to one or more of the K-wires, such as the connected K-wire. The fiducial markers may be objects, such as cubes or pyramids, that may help IGS 120 better estimate the 3-dimensional positions and orientations of the K-wires. For instance, a fiducial marker may be a cube having different pre-determined designs on each face of the cube. IGS 120 may track the position and orientation of a fiducial marker to determine the position of the K-wire to which the fiducial marker is attached. In some examples, IGS 120 may track tools attached to K-wires to determine positions and orientations of the K-wires.

In some examples, the fixation device (e.g., fixation device 402) can be modeled as a pinhole camera by a projection matrix M, relating any 3D point X to its corresponding projection q in the acquired image. The intrinsic parameters of the projection matrix describe the projection parameters from an x-ray tube to a 2D view: (u0,v0) is the principal point and f is the focal length in pixels (square pixels); the extrinsic parameters E define the orientation R and position T of the acquisition system in a world coordinate system. Thus, IGS 120 may can track a position in 2D or 3D of the tip of a K-wire.

IGS 120 (e.g., presentation unit 306 of IGS 120) may generate a visualization that includes the models of the bones superimposed on the surgical site (708). For instance, IGS 120 may generate 3D models of the bones and IGS 120 may generate the visualization such that the 3D models of the bones are superimposed on the surgical site. In another example, IGS 120 may generate 2D models of the bones and present (e.g., on display device 112A) the visualization of the 2D models of the bones superimposed on a representation of the surgical site. In this example, the visualization of the 2D models comprises showing a sagittal view and an axial view of the surgical site. By being able to see the 2D models in the sagittal and axial views, the surgeon may be able to determine the positions of the bones in 3D space. The sagittal view may be oriented in a medial or lateral direction relative to a midline of the body. The axial view may be oriented in a superior or inferior direction.

IGS 120 (e.g., presentation unit 306) may also detect changes to the positions of the external portions of the K-wires (710). For instance, IGS 120 may detect changes to the position of the connected K-wire. Based on the changes to the positions of the external portions of the K-wires, IGS 120 (e.g., presentation unit 306) may update positions of the models of the bones in the visualization to maintain correspondence between positions of the bones and the positions of the models of the bones (712). IGS 120 may continue to perform steps 710 and 712 during the surgery.

For instance, in an example where the surgery is a Lapidus surgery, the surgeon may move the first metatarsal relative to the first cuneiform bone (e.g., using features of a fixation device) so that the first metatarsal is properly aligned with the first cuneiform bone. In this example, IGS 120 may detect changes to the positions of the external portions of K-wires attached to one or more of the first metatarsal and the first cuneiform bone (which may include one or more connected K-wires). In this example, based on the changes to the external portions of the K-wires, IGS 120 may update the positions of the models of the first metatarsal and/or first cuneiform bone to maintain correspondence between the positions of the real first metatarsal and/or first cuneiform bone and the models of these bones. In other examples, the surgery may include a Chevron surgery to correct a bunion in a foot of the patient. In some examples, the surgery may include a Charcot foot stabilization surgery.

In some examples, in addition to showing models of the bones (and, in some instances. K-wires) in the visualization, IGS 120 may include other virtual objects in the visualization. For instance, in an example where the surgery is a Lapidus surgery, IGS 120 may include a virtual marker in the visualization that shows where the surgeon is to insert a surgical item (such as surgical pin, surgical screw, or drill bit) that is to pass through the first metatarsal and first cuneiform bone. That is, IGS 120 may determine, based on the registration data, an insertion point on the skin of the patient for insertion of the surgical item. The visualization may then include a virtual indication of the insertion point on the skin of the patient for insertion of the surgical item. Similar techniques may be applied to indicate insertion points for other types of surgeries. In an example where the surgery is a minimally invasive Chevron-Akin (MICA) surgery, Akin surgery, or other type of surgery, using this technique may help a surgeon to use a cutting burr to create an osteotomy at a correct location and angle.

Furthermore, in some examples, IGS 120 may track the positions of various surgical items, such as tools, pins, or other types of objects used during a surgery. Based on the positions of the surgical items, IGS 120 may include virtual representations of the surgical items in the visualization and/or include virtual objects that provide guidance for using the surgical tools. For instance, in a Lapidus surgery, IGS 120 may include a virtual object in the visualization that shows a current position of a pin as the pin is inserted through the first metatarsal of the patient. For instance, in examples where the visualization is an MR or AR visualization, the visualization may include a virtual model of a surgical pin superimposed on the model of the first metatarsal bone as the surgical pin is inserted lengthwise through the first metatarsal bone.

Certain techniques of this disclosure are described with respect to surgeries on the lower extremities (e.g., foot and ankle) of humans. However, the techniques are not so limited, and the visualization system may be used to provide virtual guidance information, including virtual guides in other types of surgeries.

Certain examples of this disclosure are described with reference to the accompanying drawings, wherein like reference numerals denote like elements. It should be understood, however, that the accompanying drawings illustrate only the various implementations described herein and are not meant to limit the scope of various technologies described herein. The drawings show and describe various examples of this disclosure. In this disclosure, numerous details are set forth. However, it will be understood by those skilled in the art that the present invention may be practiced without these details and that numerous variations or modifications from the described examples may be possible.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention. Moreover, techniques of this disclosure have generally been described with respect to human anatomy. However, the techniques of this disclosure may also be applied to animal anatomy in veterinary cases.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuitry,” as used herein may refer to any of the foregoing structures or any other structure suitable for implementation of the techniques described herein.

Various examples have been described. These and other examples are within the scope of the following claims.

1: A computer-implemented method comprising:
obtaining, during a surgery performed on a patient, one or more intraoperative images of a surgical site of the patient, wherein:
the surgical site includes a plurality of bones that are not substantially exposed through skin of the patient during the surgery,
a connected K-wire is attached to a bone of the plurality of bones,
an external portion of the connected K-wire is connected to a fixation device that is attached to the patient substantially external to the skin of the patient, and
the connected K-wire includes a respective external portion that extends outside the skin of the patient,
generating models of the bones and the connected K-wire;
determining, based at least in part on the one or more intraoperative images, one or more positions on the external portion of the connected K-wire in a real-world coordinate system;
performing a registration process that generates registration data for mapping the positions on the external portion of the connected K-wire in the real-world coordinate system with corresponding positions of a model of the connected K-wire in a virtual coordinate system;
generating a visualization that includes the models of the bones superimposed on the surgical site;
detecting changes to the positions of the external portion of the connected K-wire; and
based on the changes to the positions of the external portion of the connected K-wire, updating positions of the models of the bones in the visualization to maintain correspondence between positions of the bones and the positions of the models of the bones.

2: The computer-implemented method of claim 1, wherein:
generating the models of the bones comprises generating 3D models of the bones, and
generating the visualization comprises generating the visualization such that the 3D models of the bones are superimposed on the surgical site.

3: The computer-implemented method of claim 2, wherein generating the 3D models of the bones comprises:
obtaining preoperative 3D models of the bones;
generating intraoperative 3D models of the bones and 3D models of the connected K-wire based on the intraoperative images; and
aligning a preoperative 3D model of the connected K-wire with the preoperative 3D models of the bones based on comparison of landmarks on the preoperative 3D models of the bones and corresponding landmarks on the intraoperative 3D models of the bones.

4: The computer-implemented method of claim 1, wherein:
generating the models of the bones comprises generating 2-dimensional (2D) models of the bones, and
generating the visualization comprises generating a visualization of the 2D models of the bones superimposed on the surgical site.

5: The computer-implemented method of claim 4, wherein generating the visualization of the 2D models comprises generating a sagittal view and an axial view of the surgical site.

6: The computer-implemented method of claim 1, wherein the fixation device includes one or more features for moving the connected K-wire relative to other K-wires attached to bones of the plurality of bones.

7: The computer-implemented method of claim 1, wherein:
the fixation device includes one or more calibration objects of known sizes,
the intraoperative images include phantoms caused by the calibration objects,
the method further comprises:
determining calibration parameters based on at least one of:
a comparison of the known sizes of the one or more calibration objects and apparent sizes of the phantoms caused by the calibration objects in the intraoperative images, or
a comparison of relative positions of the one or more calibration objects of the fixation device and apparent positions of the phantoms caused by the calibration objects in the intraoperative images; and
modifying the one or more intraoperative images based on the calibration parameters, and
generating the models of the bones and the model of the connected K-wire based on the modified one or more intraoperative images.

8: The computer-implemented method of claim 1, wherein:
the surgery is a Lapidus surgery, and
updating the positions of the models of the bones in the visualization comprises moving a position of a model of a first metatarsal bone relative to a position of a model of a first cuneiform bone.

9: The computer-implemented method of claim 8, wherein:
the visualization is a Mixed Reality (MR) or Augmented Reality (AR) visualization, and
the visualization includes a virtual model of a surgical pin superimposed on the model of the first metatarsal bone as the surgical pin is inserted lengthwise through the first metatarsal bone.

10: The computer-implemented method of claim 8, further comprising:
determining, based on the registration data, an insertion point on the skin of the patient for insertion of the surgical pin,
wherein the visualization includes an indication of the insertion point on the skin of the patient for insertion of the surgical pin.

11: The computer-implemented method of claim 1, wherein generating the visualization comprises generating a Mixed Reality (MR) visualization for display on a head-mounted MR visualization device configured to allow a user to view the MR visualization and directly see a real-world environment.

12: The computer-implemented method of claim 10, wherein determining the positions of the external portion of the connected K-wire in the real-world coordinate system comprises determining, based on video data generated by sensors of the head-mounted MR visualization device, the position of the external portion of the connected K-wire in the real-world coordinate system.

13: The computer-implemented method of claim 1, wherein generating the visualization comprises generating an Augmented Reality (AR) visualization for display on a computer screen that also displays images of the real-world environment.

14: The computer-implemented method of claim 1, wherein the surgery includes one of a Chevron surgery to correct a bunion in a foot of the patient, an Akin surgery, a medial displacement calcaneal osteotomy (MDCO), a minimally invasive metatarsal osteotomy (DMMO), a transverse first metatarsal osteotomy, or a transverse fifth metatarsal osteotomy.

15: The computer-implemented method of claim 1, wherein the surgery includes a Charcot foot stabilization surgery.

16: A computing system comprising:
a memory; and
processing circuitry configured to:
obtain, during a surgery performed on a patient, one or more intraoperative images of a surgical site of the patient, wherein:
the surgical site includes a plurality of bones that are not substantially exposed through skin of the patient during the surgery,
a connected K-wire is attached to a bone of the plurality of bones,
an external portion of the connected K-wire is connected to a fixation device that is attached to the patient substantially external to the skin of the patient, and
the connected K-wire includes a respective external portion that extends outside the skin of the patient,
generate models of the bones and the connected K-wire;
determine, based at least in part on the one or more intraoperative images, one or more positions on the external portion of the connected K-wire in a real-world coordinate system;
perform a registration process that generates registration data for mapping the positions on the external portion of the connected K-wire in the real-world coordinate system with corresponding positions of a model of the connected K-wire in a virtual coordinate system;
generate a visualization that includes the models of the bones superimposed on the surgical site;
detect changes to the positions of the external portion of the connected K-wire; and
based on the changes to the positions of the external portion of the connected K-wire, update positions of the models of the bones in the visualization to maintain correspondence between positions of the bones and the positions of the models of the bones.

17: The computing system of claim 16, wherein:
the processing circuitry is configured such that, as part of generating the models of the bones, the processing circuitry generates 3D models of the bones, and
the processing circuitry is configured such that, as part of generating the visualization, the processing circuitry generates the visualization such that the 3D models of the bones are superimposed on the surgical site.

18: The computing system of claim 17, wherein the processing circuitry is configured such that, as part of generating the 3D models of the bones, the processing circuitry:
obtains preoperative 3D models of the bones;
generates intraoperative 3D models of the bones and 3D models of the connected K-wire based on the intraoperative images; and
aligns a preoperative 3D model of the connected K-wire with the preoperative 3D models of the bones based on comparison of landmarks on the preoperative 3D models of the bones and corresponding landmarks on the intraoperative 3D models of the bones.

19: The computing system of claim 16, wherein:
generate the models of the bones comprises to generate 2-dimensional (2D) models of the bones, and
generate the visualization comprises to generate a visualization of the 2D models of the bones superimposed on the surgical site.

20. (canceled)

21: The computing system of claim 16, wherein the fixation device includes one or more features for moving the connected K-wire relative to other K-wires attached to bones of the plurality of bones.

22: The computing system of claim 16, wherein:
the fixation device includes one or more calibration objects of known sizes,
the intraoperative images include phantoms caused by the calibration objects,
the processing circuitry is further configured to:
determine calibration parameters based on at least one of:
a comparison of the known sizes of the one or more calibration objects and apparent sizes of the phantoms caused by the calibration objects in the intraoperative images, or
a comparison of relative positions of the one or more calibration objects of the fixation device and apparent positions of the phantoms caused by the calibration objects in the intraoperative images; and
modify the one or more intraoperative images based on the calibration parameters, and
generate the models of the bones and the model of the connected K-wire based on the modified one or more intraoperative images.

23: The computing system of claim 16, wherein:
the surgery is a Lapidus surgery,
the processing circuitry is configured such that, as part of updating the positions of the models of the bones in the visualization, the processing circuitry moves a position of a model of a first metatarsal bone relative to a position of a model of a first cuneiform bone,
the visualization is a Mixed Reality (MR) or Augmented Reality (AR) visualization, and
the visualization includes a virtual model of a surgical pin superimposed on the model of the first metatarsal bone as the surgical pin is inserted lengthwise through the first metatarsal bone.

24. (canceled)

25: The computing system of claim 23, wherein:
the processing circuitry is configured to determine, based on the registration data, an insertion point on the skin of the patient for insertion of the surgical pin; and
the visualization includes an indication of the insertion point on the skin of the patient for insertion of the surgical pin.

26: The computing system of claim 16, wherein the processing circuitry is configured such that, as part of generating the visualization, the processing circuitry generates a Mixed Reality (MR) visualization for display on a head-mounted MR visualization device configured to allow a user to view the MR visualization and directly see a real-world environment.

27. (canceled)

28: The computing system of claim 16, wherein the processing circuitry is configured such that, as part of generating the visualization, the processing circuitry generates an Augmented Reality (AR) visualization for display on a computer screen that also displays images of the real-world environment.

29: The computer-implemented computing system of claim 16,
wherein the surgery includes one of a Chevron surgery to correct a bunion in a foot of the patient, an Akin surgery, a medial displacement calcaneal osteotomy (MDCO), a minimally invasive metatarsal osteotomy (DMMO), a transverse first metatarsal osteotomy, or a transverse fifth metatarsal osteotomy, or
wherein the surgery includes a Charcot foot stabilization surgery.

30-32. (canceled)
